(19) United States
(12) Patent Application Publication
(10) Pub. No.: US 2009/0112094 A1
(43) Pub. Date: Apr. 30, 2009

(54) Phased Apply Ultrasound With Electronically Controlled Focal Point For Assessing Bone Quality Via Acoustic Topology And Wave Transmit Functions

(75) Inventors: Yi-Xian Qin, Port Jefferson Station, NY (US); Yi Xia, Port Jefferson, NY (US); Wei Lin, Port Jefferson, NY (US)

Correspondence Address: THE FARRELL LAW FIRM, P.C., 290 Broadhollow Road, Suite 210E, Melville, NY, 11747, US

(73) Assignee: The Research Foundation of State University of New York, Albany, NY (US)

(21) Appl. No.: 12/297,079

(22) PCT Filed: Apr. 13, 2007
(86) PCT No.: PCT/US2007/009281

§ 371 Date: Oct. 14, 2008

Related U.S. Application Data
(60) Provisional application No. 60/791,642, filed on Apr. 13, 2006.
(60) Provisional application No. 60/791,644, filed on Apr. 13, 2006.

(51) Int. Cl. A61B 8/00

(52) U.S. Cl. 600/447

(57) ABSTRACT

A surface topology map technique and apparatus are disclosed for determining calcaneus thickness for imaging quantitative ultrasound measurements; improving measurement accuracy, particularly in in vivo applications.

### PRIORITY

This application claims priority to U.S. Provisional Application Ser. Nos. 60/791,642 and 60/791,644, both filed on Apr. 13, 2006.

### GOVERNMENT SUPPORT

The invention was supported, in part, by NASA Cooperative Agreement NCC9-58, NIH R01 (AR49286), and National Space Biomedical Research Institute (TD00207 and TD00405). The U.S. Government has certain rights in the invention.

### BACKGROUND

1. Field of the Invention

Ultrasound imaging allows for noninvasive, nondestructive assessment, in a real-time manner, of material density and strength of hard tissue and irregular material, in an irregular working environment, i.e., complex three dimensional shapes, non-uniform internal structures and material properties, and, in particular, complex surface topology.

2. Description of the Related Art

Distance to an object can be measured by use of ultrasound. Ultrasound Velocity (UV) is a primary acoustic property that directly relates to bone quality evaluation. See U.S. Pat. No. 4,471,785, the contents of which are incorporated herein by reference. Calculation of UV requires knowledge of a tissue thickness value in the ultrasound pathway, which is extremely difficult to determine in vivo due to irregularities of bone surface and surrounding soft tissue.

UV is a principal ultrasound parameter for bone quality assessment that relies upon a theoretical relation of bone elastic properties. Knowledge of bone thickness is very critical to accurately measure UV. However, actual thickness of bone is not directly available in vivo (under living condition) because various bones, such as the calcaneus and hip, are surrounded by soft tissue and with irregular shapes, which precludes direct measurement of bone topology. See U.S. Pat. Nos. 5,921,929, 5,785,656, 5,771,310, 5,603,325, 5,218,963, 5,197,475 and 5,587,533, the contents of each of which are incorporated herein by reference. Thus, accuracy of UV measurements will be undermined if only a single preset thickness is used, as done in many commercial Quantitative Ultrasound (QUS) systems. Prior studies that used a preset heel thickness to calculate UV using a commercially available single index ultrasound device (e.g., CUBA system, or Lunar Achilles) overestimated the value by up to 78 ms over 1753 m/s UV value (approximately 5% error) compared to the UV using measured heel dimension with soft tissue removed. See Hausler et al. (1997). Such overestimation spans the UV differences between normal and osteoporotic bones, creating diagnostic inaccuracy. See U.S. Pat. Nos. 6,799,066, 6,666,833, 6,231,958, and 6,205,348, the contents of each of which are incorporated herein by reference.

Accordingly, more efficient scanning is required, particularly at skeletal soft tissue sites. There is also a need to more rapidly measure and generate bone structural image in a large and deep tissue area, and to extend such rapid measurement from two-dimensional to three-dimensional imaging. To address this need, the present invention provides an ultrasonic phased array acoustic scanning apparatus and method that utilizes phased arrays of two-dimensional elements, wherein emissions are provided having different, electronically controlled delays that generate a focused ultrasonic beam via programming controls.

In addition, osteoporosis and osteopenia are major health issues affecting aging people. (QUS) provides information regarding bone structure and bone mineral density, useful for diagnosis of osteoporosis and prediction of fracture risk. QUS is easy to use, portable, inexpensive and relatively accurate. Further, QUS does not create a radiation exposure risk.

### SUMMARY OF THE INVENTION

According to a preferred embodiment of the present invention, an apparatus and method are provided for quantitative ultrasound measurement for non-invasively assessment of bone quality, to address the shortcomings of conventional systems.

An aspect of the present invention provides a bone Surface Topology Map (STM) that determines bone thickness at the point of ultrasound measurement, thereby enhancing velocity measurement accuracy.

Another aspect of the present invention provides a phased array ultrasound scanning apparatus that uses multi-element transducers, utilizing programming routines to electronically control the focal points. Each element of the transducers is connected to a different electronic channel, and each element can be activated at varied delayed times. Electronic delay is applied to each electronic channel when emitting and receiving the signal to/from the transducer elements. The combination of the phased delay pulses will form a spatial wave front designed for confocal the ultrasound beam converged at the focal zone, maximizing efficiency of the ultrasound scan.

### BRIEF DESCRIPTION OF THE FIGURES

The above and other objects, features and advantages of certain exemplary embodiments of the present invention will be more apparent from the following detailed description taken in conjunction with the accompanying drawings, in which:

FIGS. 1a-b show confocal imaging via a two-dimensional phased array matrix transducer design of the present invention;

FIGS. 2a-c show regions of interest in QUS measurements and CT measurements;

FIGS. 3a-b show a calibration block with a stepwise surface and a corresponding STM reconstructed step surface;

FIGS. 4a-c show STM images compared with corresponding CT images;

FIG. 5 is a block diagram outlining interoperability of an ultrasound scanning system of the present invention;

FIG. 6 is a flowchart showing operation of the ultrasound scanning system signal processing and calculation of UV, ultrasound attenuation number and broadband ultrasound attenuation;

FIG. 7 is a flow chart describing a bone surface mapping program; and

FIGS. 8a-b show US transmitting and surface detection, with time detection.

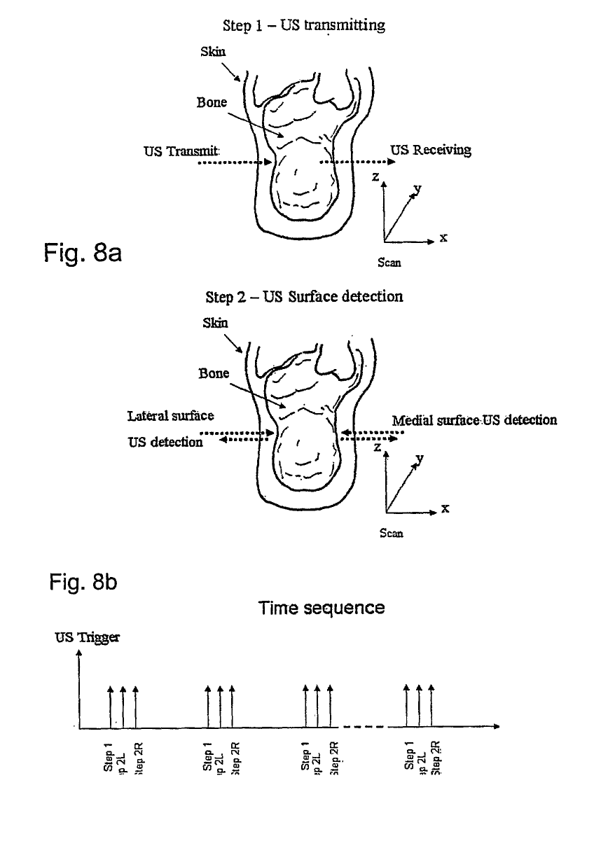

### DESCRIPTION OF THE PREFERRED EMBODIMENTS

A description of detailed construction of preferred embodiments is provided to assist in a comprehensive understanding of exemplary embodiments of the invention. Accordingly, those of ordinary skill in the art will recognize that various changes and modifications of the embodiments described herein can be made without departing from the scope and spirit of the invention. Descriptions of well-known functions and constructions are omitted for clarity and conciseness.

In the present invention, a Surface Topology Map (STM) reflects ultrasound waves from bone surfaces, such as the calcaneus, to determine spatial positions relative to ultrasound transducers at medial and lateral sides. The thickness of bone, delineated by the spacing between the two surfaces, is calculated from a position difference between the two surfaces. Importantly, corresponding surface points along a medial-lateral ultrasound wave pathway are unique with a spatial resolution of scan steps. The imaging QUS system has an inherent capability to interrogate bone topology, such as the calcaneus, using high-resolution pixel mapping, allowing bone surface position to be measured at a pixel level. A three-dimensional topology map of the calcaneus surface is reconstructed using the surface mapping technique. In addition, a two-dimensional map of the calcaneus thickness in the medial-lateral orientation is rendered from the three-dimensional topology and used for accurate calculation of UV.

In a preferred embodiment, a Scanning Confocal Acoustic Diagnostic (SCAD) system (Xia et al., 2005) is utilized to implement the technique. The SCAD system is a confocal imaging QUS system having a computer-controlled two-dimensional scanner unit with a pair of focused transducers (1 MHz, Panametrics V303). The transducers are coaxially aligned to each other and are connected to a custom-made pulser/receiver control unit, which can work under both transmission mode and pulse echo mode.

In a preferred embodiment, a scan controller is provided to obtain ultrasound measurements by sending an ultrasound signal from a first, or transmitting, transducer that is positioned at one side of an object to be scanned to a second, or receiving transducer, positioned at an opposite side of the object. The transmitter/receiver transducer pair is focused and converged at the confocal region/point, with a separation distance of approximately twice the focal length.

The transducer pairs are specially designed piezoelectric ultrasound transducers that operate in a frequency range between approximately 0.2 MHz and 20 MHz. The transducers preferably provide a 25 mm diameter, with a surface mechanical lens shaped to generate ultrasound focal length between 25 mm and 75 mm, depending on tissue thickness, with a focal point of approximately 1-2 mm in diameter. The amplified signal is sent to the transmitting transducer, which emits the amplified ultrasonic wave (in general, a pulse having a pulse width between 0.1 and 5.0 microseconds) through bone to be detected by the receiving transducer. The ultrasound pulse thus radiated by the transmitting transducer is transmitted into the propagation medium. In a preferred embodiment, the measurement is carried out in an immersion mode to maximize coupling between the radiation source and subject bone. The receiving transducer converts the detected waveform into an electrical signal, which is amplified by a pre-amplifier unit.

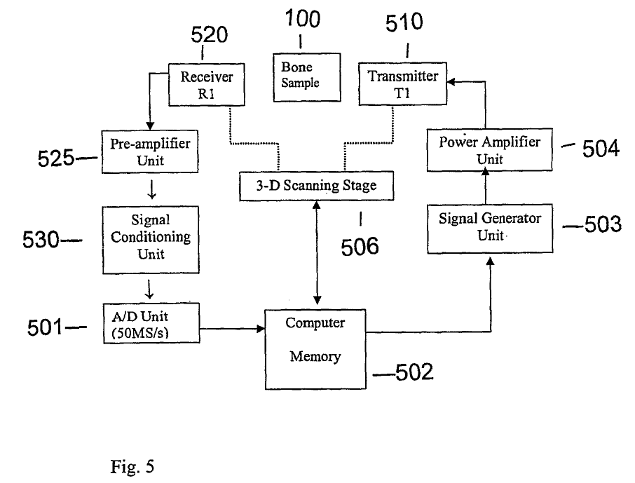

In the present invention, signal-filtering, gain control and other pre-processing tasks are performed by signal conditioning and an analog-to-digital conversion receives analog signals acquired in real time from the signal conditioning, and provides digital signal outputs to the embedded computer. These functions are controlled by a custom-designed microprocessor, preferably based on 8051 microcontroller or customer preprogrammed Field Programmable Gate Array (FPGA), to control a signal generator by providing waveform data thereto, and, in a preferred embodiment, setting a rate of pulse or tone burst and other control signals; independently controlling movement (range and speed) of a three-dimensional scanning stage to perform a three-dimensional scan of the specimen (either discretely or continuously) including coordinating movement of the three-dimensional scanning stage to allow the three-dimensional scanning stage to perform a two-dimensional scan of the x-y plane of the specimen along a z axis; processing the received digitized ultrasound signals from the A/D in time and frequency domain to generate ultrasound images; reconstructing the three-dimensional images or other representing forms of the sample data; calculating the propagation times of the signals transmitted through the specimen; calculating the elastic modulus (E) (e.g., MP); calculating the bone thickness passed through at the measurement location; and calculation of surface topology at resolution of less than 100 microns, as discussed in further detail in regard to FIG. 5, below. Calculation of propagation velocity of ultrasound in transmission (UV), calculation of the ultrasound attenuation number (ATT) (in dB), calculation of the broadband ultrasound attenuation (BUA) (in dB/MHz), and calculation of Bone Mineral Density (BMD) and stiffness is also preferably performed.

The ultrasound signal received from transducers is digitized at 25 MHz using high-speed digitizer (Gagescope, CS1250) installed in a receiving computer, such as a Dell Dimension workstation, running control software written in C++ language. STM was performed under pulse echo mode. At each scanner point, an ultrasound signal was sent toward the bone surface and the reflected echo wave was recorded. This process was repeated over a maximum 40×40 mm area at 0.5 mm scanning resolution. The recorded waves were analyzed using custom written software based on the Time-Of-Flight (TOF) algorithm.

STM software was developed based on TOF using Labview (National Instruments) and Matlab (ver. 6.5; The MathWorks INC.). The algorithm calculates TOF (Δt) utilizing Equation (1):

2L_{tb}=V_{w}*Δt (1)

where V_{w} is wave speed in water and Δt is time used before the reflected wave is recorded.

Distances between lateral side transducers and lateral sample surface (L_{tbl}) and between medial side transducers and medial sample surface (L_{tbm}) can both be determined using Equation (1). If the distance between two ultrasound transducers (L) is fixed, sample thickness at each scanning point (d) can be determined by subtract L_{tbl} and L_{tbm} from L, in accordance with Equation (2):

D=L−Ltbl−L_{tbm} (2)

Information regarding d is calculated at each imaging pixel (dᵢⱼ) and the velocity is calculated utilizing Equation, (3):

$${V}_{ij} = \frac{{V}_{w} {d}_{ij}}{{d}_{ij} + {V}_{w} Δτ} \qquad ( 3 )$$

where V_{w} is a wave speed in water and Δτ is the arrival time difference between the reference wave through water (without the bone sample in the water pathway) and the sample wave through the bone tissue, with both measured in a transmission mode for calculating UV.

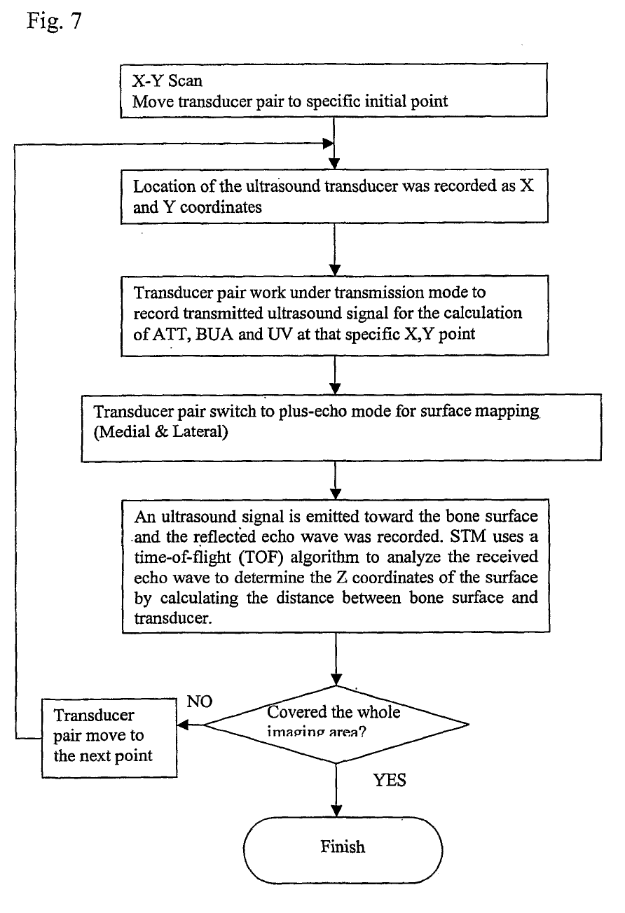

The relative position of bone surface to the transducer at each scanning point can be determined to form a three-dimensional topology map for medial and lateral sides. The distance between the medial and lateral surface was the thickness of the calcaneus. With the bone in the same position, the system is switched to the transmission mode and the transmitted ultrasound wave is recorded over the same scanning are with the same resolution. The thickness determined by STM at each measurement is used to calculate the UV value at the same point. A flow chart describing a preferred bone surface mapping program is provided at FIG. 7.

QUS measurement has been used for non-invasively assessing bone quality, i.e. density and strength. UV is a primary acoustic property that directly relates to bone quality evaluation. However, the calculation of UV requires the tissue thickness value in the ultrasound pathway, which is extremely difficult to be determined in vivo due to irregularities of bone surface and surrounding soft tissue. Further to the studies of Hausler et al. discussed above, prior in vivo research performed by Chappard et al. (2000) indicates that UV error introduced by unknown soft tissue thickness and properties can be 3-20 times higher than the UV where short-term precision and thickness correction for overlying soft tissue at calcaneus site was introduced.

Correction of bone thickness at the measurement point is also important for confocal imaging QUA techniques where the UV value obtained for each imaging pixel has a thickness specific to a certain measurement point. Considering, for example, the irregular shape of calcaneus bone, the thickness variation could reach 5-8 mm throughout the region of the scanned area. To accurately determine the thickness of the bones such as the calcaneus and enhance the accuracy of velocity measurements, the present invention provides an STM technique.

The present invention provides a bone STM method that accurately determines bone thickness at the point of ultrasound measurement and enhances the accuracy of velocity measurement. Results from a study of twenty-five calcaneus human cadaver samples show that irregular medial and lateral surfaces of calcaneus can be accurately determined using STM. UV calculated based on STM determined thickness has significantly better correlation to BMD (r²=0.56), with Bone Volume fraction (BV/TV) (r²=0.52) and bone modulus (r²=0.48) than UV without use of the inventive technique (P<0.05). The later correction coefficients were r²=0.41 for BMD, r²=0.42 for BV/TV, and r²=0.34 for modulus. The results indicate that STM technique in scanning ultrasound can accurately determine calcaneus bone thickness, thereby enhancing UV accuracy in bone property measurement. In addition, the present invention is non-invasive and easily incorporated into in vivo clinical diagnostic devices.

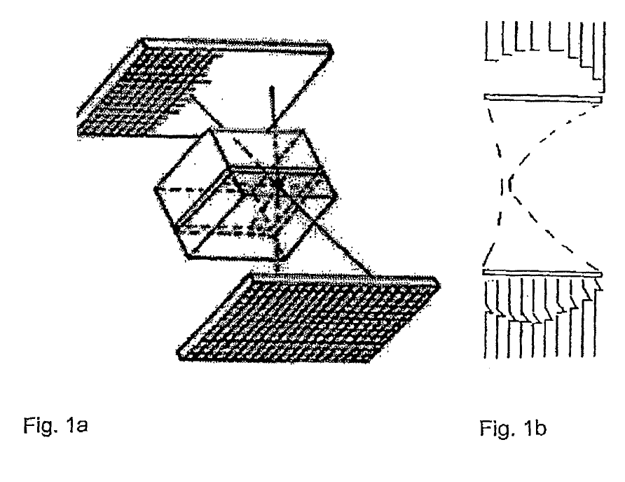

FIGS. 1a and 1b show a confocal mode achieved by inventive phased array two-dimensional matrix transducer design and soft code development. Phased array ultrasound scanning involves multi-element transducers made up of piezoelectric elements each connected to a different electronic channel. Each element can be activated at any particular delay time. An electronic delay is preferably applied to each electronic channel when emitting and receiving the signal to/from transducer elements. The combination of the phased delay pulses form a spatial wave front designed to confocal converge the ultrasound beam at the focal zone. It is preferable to utilize probes having very low acoustic and electric cross coupling between the elements, allowing each element to be independently fired.

The phased array ultrasound uses multi-element transducers, and the focal points are preferably controlled electronically via programming, and each transducer element is connected to a different electronic channel. Each element is preferably activated at any delayed time, and an electronic delay is applied to each electronic channel when emitting and receiving the signal to/from the transducer elements.

Electronic focusing and steering is utilized in a two-dimensional phased array to focus the beam by applying systematic delays to the different elements. The receiving phased array decodes the focused beam by mirror technique incorporating with the transmitter array, as shown in FIG. 1. The transmission and receiving arrays are relatively positioned in a stationary manner. To generate a spatial focal point and focal plane, both transmission and receiving arrays are coded with spatial and time sequence of the signals. Coding patterns for both arrays are mirrored by the focal plane. The transmitter array provides advantages of fast scanning of the specimen, wherein only several elements need be simultaneously excited. The scan preferably combines electronic focusing and steering. Delay laws are utilized to deflect the beam, and application to different elements of the two-dimensional matrix array allows for three-dimensional beam steering, in which activation of transmission and receiving signals are programmed with a designed time sequence that can send or receive ultrasound signals to and from at the focal points and the focal plane.

The beams are also preferably generated and received using focal laws, in which software models the programs to spatially control the confocal points and scanning. The ultrasound energy is generated at the focal points by a designed spatial time sequence ultrasound transmission program, which regulates the excitation sequence of each element on the array, by which the ultrasound wave front can generate virtual convergence plane that can focus the ultrasound energy at the focal points. Scan times for a 100×100 mm² scan array with 0.5 mm resolution are approximately 30 seconds. Table I lists bone properties (mean, standard deviation, range) measured from QUS, Dual-Energy X-Ray Absorptiometry (DEXA), μCT and mechanical testing (N=25), separated into ultrasound, bone density, architecture and mechanical properties categories.

TABLE 1

| Ultrasound | | | |
|---|---|---|---|
| BUA | 52.2 | 20.7 | 15.3-98.7 |
| UV(FIX) | 1502 | 27 | 1451-1566 |
| UV(STM) | 1465 | 35 | 1412-1533 |
| Bone Density | | | |
| BMD | 0.41 | 0.35 | 0.01-0.88 |
| Architecture | | | |
| BV/TV | 0.15 | 0.06 | 0.04-0.25 |
| SMI | 2.24 | 0.59 | 1.35-3.38 |
| Conn. | 3.01 | 1.55 | 0.49-5.63 |
| Tb.N | 1.31 | 0.23 | 0.87-1.67 |
| Tb.Th | 0.19 | 0.02 | 0.13-0.22 |
| Tb.Sp | 0.74 | 0.16 | 0.53-1.13 |
| Mechanical | | | |
| Modulus | 386.6 | 206.8 | 60.5-810.9 |
| Yield | 2.77 | 1.84 | 0.4-7.57 |
| Ultimate | 2.81 | 1.89 | 0.4-7.8 |

Linear regression tests among QUS, DEXA, CT and mechanical test showed that UV determined by using STM (V_{STM}) rendered higher correlations with bone material and structural indices when compared to UV with fixed thickness (V_{FIX}). V_{STM} showed much better correlation to BMD (r²=0.56) and modulus (r²=0.48) compared to the V_{FIX} (BMD: r²=0.41; Modulus: r²=0.34). The nBUA value determined using STM was also highly correlated to BMD (r²=0.74) and modulus (r²=0.62), comparable to the correlation result for BUA (BMD: r²=0.76; Modulus: r²=0.64). Table 2, below, provides correlations (r² value) (N=25) among UV, BMD and microarchitectural features, with comparison between surface mapping (STM) and non-surface mapping technique (FIX).

TABLE 2

| | BDM | BV/TV | SMI | Conn. | Tb.N | Tb.Th | Tb.Sp | Modulus |
|---|---|---|---|---|---|---|---|---|
| UV (STM) | 0.56* | 0.52* | 0.38* | 0.48* | 0.44* | 0.38* | −0.45* | 0.48* |
| UV (FIX) | 0.41* | 0.42* | 0.24* | 0.42* | 0.49* | 0.34* | −0.44* | 0.34* |

*p < 0.01

The present invention provides a more accurate estimation of the calcaneus thickness in a pixel-based manner. The hardware and software modulation on SCAD system required by STM delivers accurate results. Moreover, the SCAD system shares many features with conventional commercial QUS systems used in in vivo clinical applications.

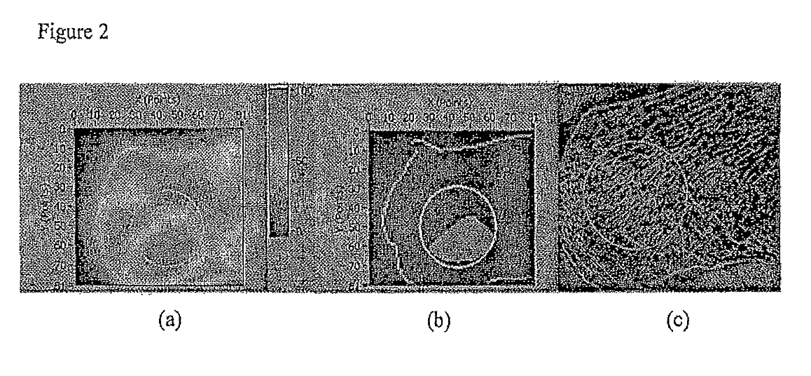

FIG. 2 shows Regions Of Interest (ROIs) in QUS measurements and CT measurements. The ultrasound attenuation number (ATT) is calculated as the ratio of two ultrasound response signals, the reference signal discussed above and a bone specimen signal. The ratio is calculated at each scanning point of the bone specimen as Equation (4):

ATT=10* LOG {(energy of reference signal)/(energy of bone signal)} (4)

In FIG. 2, an automatic irregular ROI searching algorithm searched for a lowest ATT pixels on the ATT image of FIG. 2a, inside a middle of a posterior part of the calcaneus (indicated by the circle in FIG. 2a). A 500 pixel ROI was automatically detected by the algorithm with the white area inside the circle, as indicated by the picked pixel in FIG. 2b. A similar circular ROI was manually determined on the CT image in FIG. 2c, corresponding to the ROI searching area in QUS, for measurement of micro structural indexes.

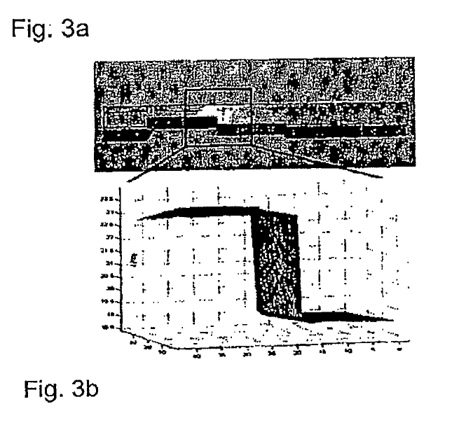

FIGS. 3a-b shows a calibration block with a stepwise surface and a corresponding STM reconstructed step surface. An aluminum calibration block with stepwise surface is shown in FIG. 3a and a corresponding STM reconstructed step surface is shown in FIG. 3b.

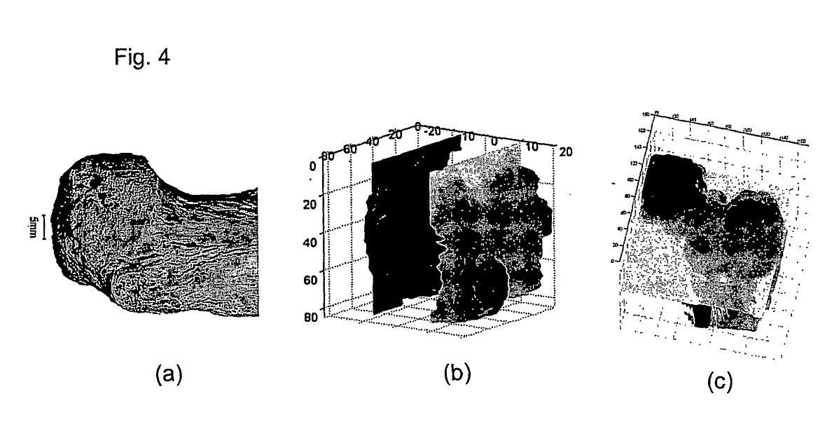

FIGS. 4a-c show STM images compared with corresponding CT images. STM reconstructed calcaneus surfaces shown in FIG. 4b correspond to the actual surfaces measured by vivaCT in FIG. 4a (bottom view, with medial surface on the right). An angle view of the same STM reconstructed calcaneus is provided in FIG. 4c with medial surface at the front and lateral surface at the back, clearly showing anatomic features of the calcaneus with the protruded medial process at the posterior part of the calcaneus and sustentaculum and a front part of the calcaneus, wherein a three-dimensional image can be represented as a two-dimensional thickness image for matched calculation of UV at each imaging pixel.

FIG. 4 shows that the stepwise surface of the calibration block depicted by the STM technique provides good similarity. In FIG. 4, STM measured step thickness on the aluminum calibration block surface was 4.2 mm, found to be in good accordance with the actual step thickness of 4.3 mm. FIG. 4b shows three-dimensional reconstructed surfaces of a representative calcaneus sample and corresponding three-dimensional surfaces measured by μCT (FIG. 4a). The irregular surfaces of the calcaneus could be clearly depicted using STM, with the curved medial surface and relative flat lateral surface (FIG. 4b), which corresponds to the actual surface curvature of the calcaneus measured by ACT. Bone thickness is determined at each mapping point. Studies utilizing the present invention provided a three-dimensional shape of the calcaneus sample determined by using STM technique, and found the three-dimensional image corresponds to the actual calcaneus shape reconstructed form CT scanning.

Current in vivo measurement of UV uses a preset heel width or individual heel width measured using contact method. See Lauguer et al., 1996; Laugier 2002, U.S. 2001/0031922 A1, U.S. 2003/0067249 A1, U.S. 2002/0145941 A1, U.S. 2005/0160817 A1 and U.S. Pat. No. 6,585,649, the contents of each of which are incorporated herein by reference. The heel width is not the width of calcaneus bone, but is the width of calcaneus with surrounding soft tissue, which introduces measurement error and diminishes the inter-individual differences, thereby making cross-sectional comparison between individuals using UV parameter less favorable. The present invention accurately measures UV using STM technique, providing a more robust and favorable parameter. In addition, STM technique allows monitoring of the three-dimensional position of the calcaneus, regardless of translation and rotation of calcaneus bone. Position change of the medial and lateral surfaces can be potentially detected by the STM technique and is utilized to identify the correct position of the calcaneus. In contrast, previous research has shown that the effects of foot positioning were more pronounced compared to other factors affecting in vivo precision. See Evans et al., 1995. STM implementation for in vivo measurements with real-time compatibilities reduces foot-positioning error.

FIG. 5 is a block diagram outlining interoperability of an ultrasound scanning system of the present invention. In FIG. 5, an A/D Unit 501 and a three-dimensional scanning stage 506 provide input to memory 502, which controls signal generating unit 503, the signal from which is amplified at power amplifying unit 504. Transmitters 510 and 520 are positioned on opposite sides of bone sample 100, and for control by the three-dimensional scanning stage 506. Output from receiver 520 is provided to pre-amplifying unit 525 for processing by signal conditioning unit 530.

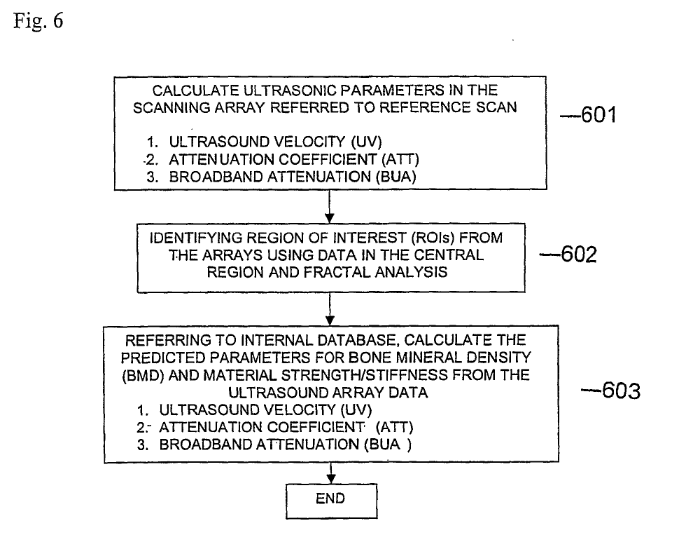

FIG. 6 is a flowchart showing operation of the ultrasound scanning system signal processing phase, for calculation of UV, ATT and BUA. At step 601, ultrasonic parameters are calculated in the scanning array referred to the reference scan. At step 602, ROI's are identified from arrays using data in the central region and fractal analysis, and, at step 603, an internal database is referred to for calculation of predicted parameters of BMD and material strength/stiffness received from the ultrasound array data.

While the present invention has been particularly shown and described with reference to preferred embodiments thereof, it will be understood by those skilled in the art that various changes in form and details may be made therein without departing from the scope of the invention encompassed by the appended claims.

1-15. (canceled)

16. A method for determining bone strength, the method comprising:
determining, via reflection from bone surfaces of first ultrasound waves transmitted by coupled phased arrays of paired ultrasound transmitters/receivers, relative positions of opposite bone surfaces;
creating a three-dimensional bone map utilizing the reflected first ultrasound waves;
transmitting a second ultrasound signal via a transmitter of the transmitter pair positioned at a first bone surface;
receiving the second signal at an opposite bone surface by a receiver of an ultrasound transmitter/receiver pair;
determining bone density by considering delay of the second ultrasound signal; and
combining a plurality of determined relative positions of opposite bone surfaces with the determined bone density.

17. The method of claim 16, wherein systematic electronic delays are applied to each of a plurality of channels for emitting and receiving ultrasound signals communicated between the ultrasound transducers of the transmitter/receiver pairs.

18. The method of claim 17, wherein the systematic electronic delays create phased delayed pulses.

19. The method of claim 18, wherein the transmitter/receiver pairs include multi-element transducers for focusing the ultrasound beam on a focal point.

20. The method of claim 18, wherein a phased array receivers decode the focused beam by mirror technique of the phased array transmitters.

21. The method of claim 19, wherein the phased delayed pulses are combined to form a spatial wave front converging at the focal point of the bone surface.

22. The method of claim 18, wherein electronic focusing and steering is utilized in a two-dimensional phased array to focus the ultrasound signals.

23. The method of claim 18, wherein the signal delay is determined via a time of flight of the ultrasound signals determined by:
2L_{tb}=V_{w}*Δt,
wherein V_{w} is wave speed.

24. The method of claim 18, wherein, if a distance between two ultrasound transducers (L) is fixed, bone thickness is determined by:
D=L−L_{tbl}−L_{tbm}.

25. The method of claim 24, wherein d is calculated at each imaging pixel (dᵢⱼ) and velocity is calculated by:
$${V}_{ij} = \frac{{V}_{w} {d}_{ij}}{{d}_{ij} + {V}_{w} Δτ} ,$$
wherein V_{w} is a wave speed and Δτ is the arrival time difference.

26. The method of claim 18, wherein a surface topology is developed to provide an in vivo assessment of bone quality.

27. An apparatus for medical assessment, the apparatus comprising:
a plurality of coupled ultrasound transmitters/receivers arranged in phased arrays to determine relative positions of opposite surfaces of a bone positioned between the phased arrays to create a three-dimensional bone surface map utilizing reflected ultrasound waves, wherein
ultrasound signals transmitted through the bone are received by a receiver coupled to a transmitter of a predetermined transmitter/receiver pair, to determine a signal delay for assessing bone density, and
the determined relative positions of the opposite bone surfaces are combined with the assessed bone density to calculate bone quality.

28. A method for determining bone quality, the method comprising:
determining, via reflection from bone surfaces of waves transmitted by coupled phased arrays of paired transmitters/receivers, relative positions of surfaces on opposite sides of the bone;
creating a three-dimensional bone surface map utilizing a plurality of the relative positions of the opposite surfaces;
transmitting a signal through the bone via a transmitter of a transmitter/receiver pair;
receiving the signal by the receiver of the transmitter/receiver pair;
determining bone density by considering signal delay between the transmitter and receiver of the transmitter/receiver pair; and
combining the determined relative positions with the determined bone density to determine bone quality.
